(19) United States
(12) Patent Application Publication
(10) Pub. No.: US 2010/0136666 A1
(43) Pub. Date: Jun. 3, 2010

(54) GARBAGE DISPOSAL SYSTEM AND MAINTENANCE SCHEDULE CREATING METHOD OF GARBAGE DISPOSAL SYSTEM

(75) Inventors: Shigemitsu Kobayashi, Chiba-ken (JP); Mitsuyo Abe, Tokyo (JP)

Correspondence Address: DLA PIPER LLP US, P. O. BOX 2758, RESTON, VA, 20195, US

(73) Assignees: JR EAST URBAN DEVELOPMENT CORPORATION, Shibuya-ku, Tokyo (JP); THINKING JAPAN CO., LTD., Toshima-ku, Tokyo (JP)

(21) Appl. No.: 11/997,731

(22) PCT Filed: Jul. 28, 2006
(86) PCT No.: PCT/JP2006/314962

§ 371 Date: Feb. 1, 2008

(30) Foreign Application Priority Data
Aug. 4, 2005 (JP) 2005-226141

(51) Int. Cl. C12M 1/02; C12S 3/00

(52) U.S. Cl. 435/262; 435/290.2

(57) ABSTRACT

A technique of making a maintenance program concerning a garbage disposal system by analyzing at least the weight of garbage brought in by users at least for one week while disposing of the garbage. Before garbage to be disposed of is inputted, the identification information on the user and the date and time of when the garbage is inputted is entered. Only when the identification information agrees with the pre-registered information, the inlet is opened. The weight of the garbage inputted through the inlet is detected with a sensor. The data on the detected weight for each user is stored in a memory. According to the stored data on the weights for one week, an inspection schedule and a bacteria adjustment/supplement schedule of the stirring mechanism and deodorizing mechanism are determined.

### TECHNICAL FIELD

The present invention mainly relates to a maintenance schedule creation technique of a garbage disposal system in which food waste is decomposed into carbon dioxide gas and water by using microorganisms.

### BACKGROUND ART

In the natural world, garbage is weathered over a long time period and decomposed by bacteria such as soil bacterium. There is a garbage disposal system configured, by applying the garbage decomposition process in the natural world, so as to be capable of decomposing and disposing specifically food waste within a short time period (refer to Patent Document 1 (Japanese Unexamined Patent Application Publication No. 9-1112), for example).

Further, Koai Sangyo Co. developed, as KOAI bacteria (trademark pending), bacteria in which based on Bacillus bacteria widely living in the natural world like soil, dried grass and air, facultative anaerobic bacteria (amphoteric) and aerobic bacteria of cellulose decomposing bacteria, animal-fat decomposing bacteria, vegetable-fat decomposing bacteria, mineral-oil decomposing bacteria, protein decomposing bacteria, lignin decomposing bacteria (wood fungus), and the like are caused to symbiotically live, depending on an object to be disposed (refer to Non-Patent Document 1 (Nikkei newspaper, issued on Jan. 9, 2004), for example).

By inputting this bacteria into a garbage disposal system, it is possible to decompose food wastes mainly including garbage but also including polyethylene and polystyrene, and it is possible to achieve, for example, a volume reduction rate of 98% or higher within 24 hours even in the case of food waste packed in a plastic bag, case or package.

When introducing such a garbage disposal system, it is necessary to firstly conduct a survey on the actual condition of the amount and components of food wastes (referred to as “garbage” in a generic term) such as garbage, left-over meals, prepared food in packages and wrapped food materials, and determine a place to install the garbage disposal system, processing ability, the combination of used bacteria, and a maintenance program thereof.

The maintenance program is a schedule related to the process and maintenance of the garbage disposal system such as the combination of used bacteria, the amount of used bacteria, a set temperature inside a stirring tank, and the inspection, regulation and repair of the garbage disposal system.

It has been impossible to create an appropriate maintenance program unless, in the survey on the actual condition before introduction of the system, the amount and components of the garbage discharged by potential users in the installation area are surveyed for a long period of about one year and estimated. The amount and components of the discharged garbage are various. For example, the amount and components of the discharged garbage change depending on business types such as restaurants, department stores, convenient stores and stations, business scales and areas, and also change even in one day. The amount and components also change dramatically depending on days of the week, seasons, and weather conditions. Therefore, there is a problem that creation of the maintenance schedule takes time, labor and cost.

[Patent Document 1] Japanese Unexamined Patent Application Publication No. 9-1112

[Non-patent Document 1] Nikkei newspaper, issued on Jan. 9, 2004

### DISCLOSURE OF THE INVENTION

### Problem the Invention is Intended to Solve

The present invention provides a technique for creating a maintenance program related to a garbage disposal system by analyzing at least the weight of garbage brought in by users for at least one week while disposing of garbage.

### Means for Solving the Problem

In a first aspect of the present invention, a garbage disposal system configured to dispose of garbage by a decomposing action of bacteria comprises: a data entry unit to input identification information of a user and a date on which garbage is dumped in; an opening/closing unit configured to open a dumping inlet only in a case where the identification information coincides with pre-registered information; a sensor configured to detect a weight of garbage dumped in through the dumping inlet; a stirring mechanism configured to stir dumped-in garbage; a deodorizing machine configured to deodorize odor from the garbage; a memory to accumulate data of the weight detected by the sensor for each of the users recognized by the data entry unit; and a processor configured to determine a schedule for inspection of the stirring mechanism and the deodorizing machine, and a schedule for regulation and replenishment of the bacteria, based on the data of the weight of at least one week accumulated in the memory.

In a second aspect of the present invention, a garbage disposal system configured to dispose of garbage by a decomposing action of bacteria comprises: a data entry unit to input identification information of a user and a date on which garbage is dumped in; an opening/closing unit configured to open a dumping inlet only in a case where the identification information coincides with pre-registered information; a sensor configured to detect a weight of garbage dumped in through the dumping inlet; a stirring mechanism configured to stir the dumped-in garbage; a deodorizing machine configured to deodorize odor from the garbage; a garbage data file to accumulate data of the weight detected by the sensor for each of the users recognized by the data entry unit; a maintenance program file configured to previously store a schedule for inspection of the stirring mechanism and the deodorizing machine, and a schedule for regulation and replenishment of the bacteria, in a manner separated into types in accordance with a dumping date and weight of garbage; and a maintenance processor configured to determine a schedule for next inspection of the stirring mechanism and the deodorizing machine, and a schedule for next regulation and replenishment of the bacteria, based on data of the garbage dumping date of at least one week inputted into the data entry unit, and data of the weight of at least one week accumulated in the garbage data file, with reference to the maintenance program file.

In a third aspect of the present invention, a garbage disposal system configured to dispose of garbage by a decomposing action of bacteria comprises: a garbage disposal machine including a data entry unit to input identification information of a user and a date on which garbage is dumped in, an opening/closing unit configured to open a dumping inlet only in a case where the identification information coincides with pre-registered information, a sensor configured to detect a weight of garbage dumped in through the dumping inlet, a stirring mechanism configured to stir dumped-in garbage, and a deodorizing machine configured to deodorize odor from the garbage; a garbage data file to accumulate, for a predetermined time period, data of the weight detected by the sensor for each of the users recognized by the data entry unit; a user memory file to accumulate business type and scale of the user; and a processor configured to create a schedule for inspection of the stirring mechanism and the deodorizing machine, and a schedule for regulation and replenishment of the bacteria, based on data of a garbage dumping date of at least one week inputted into the data entry unit, and data of the weight of at least one week accumulated in the garbage data file, in a manner separated types in accordance with the business type and scale of the user accumulated in the user memory file.

In a fourth aspect of the present invention, a garbage disposal system configured to dispose of garbage by a decomposing action of bacteria comprises: a data entry unit to input identification information of a user and a date on which garbage is dumped in; an opening/closing unit configured to open a dumping inlet only in a case where the identification information coincides with pre-registered information; a stirring mechanism configured to stir dumped-in garbage with a motor; a deodorizing machine configured to deodorize odor from the garbage; a sensor configured to detect a weight of the garbage dumped in through the dumping inlet and a load of the motor: a garbage data file to accumulate data of the weight and load detected by the sensor for each of the users recognized by the data entry unit: and a processor configured to determine a schedule for inspection of the stirring mechanism and the deodorizing machine, and a schedule for regulation and replenishment of the bacteria, based on the data of the weight and load of at least one week accumulated in the garbage data file.

In a fifth aspect of the present invention, a garbage disposal system comprises: first and second garbage disposal systems, each including a stirring mechanism configured to stir garbage with a motor, a load sensor configured to detect a load of the motor, and a deodorizing machine configured to deodorize odor from garbage and disposing of garbage by a decomposing action of bacteria; a data entry unit to input identification information of a user and a date on which garbage is dumped in, an opening/closing unit configured to open a clumping inlet only in a case where the identification information coincides with pre-registered information; a weight sensor configured to detect a weight of garbage dumped in through the dumping inlet; a crusher configured to crush the garbage dumped in through the dumping inlet; a deodorizing machine configured to deodorize odor from the garbage; a switching valve to convey the garbage crushed by the crusher to the first and second garbage disposal systems; a garbage data file to accumulate data of the load detected by the load sensor and data of the weight detected by the weight sensor, respectively; and a processor configured to control the switching valve based on the data of the load and weight accumulated in the garbage data file, substantially equalize the weights of the garbage disposed of by the first and second garbage disposal systems and the loads of the motors and determine a schedule for inspection of the stirring mechanism and the deodorizing machine and schedule for regulation and replenishment of the bacteria based on the data of the weight of at least one week accumulated in the garbage data file.

In a sixth aspect of the present invention, a maintenance schedule creating method of a garbage disposal system comprising a stirring mechanism configured to stir dumped-in garbage together with bacteria and a deodorizing machine configured to deodorize odor from the garbage, the maintenance schedule creating method comprising: before dumping in garbage to be disposed of, inputting identification information of a user and a date on which the garbage is dumped in; opening a dumping inlet only in a case where the identification information coincides with pre-registered information; detecting, with a sensor, a weight of the garbage dumped in through the dumping inlet; accumulating data of the detected weight into a memory for each of the users; and determining a schedule for inspection of the stirring mechanism and the deodorizing machine, and a schedule for regulation and replenishment of the bacteria, based on the accumulated data of the weight of at least one week.

In a seventh aspect of the present invention, a maintenance schedule creating method of a garbage disposal system comprising a stirring mechanism configured to stir dumped-in garbage together with bacteria and a deodorizing machine configured to deodorize odor from the garbage, the maintenance schedule creating method comprising: previously recording a schedule for inspection of the stirring mechanism and the deodorizing machine, and a schedule for regulation and replenishment of the bacteria into a maintenance program file, in a manner separated into types in accordance with a garbage dumping date and weight; before dumping in garbage to be disposed of, inputting identification information of a user and a date on which the garbage is dumped in; opening a dumping inlet only in a case where the identification information coincides with pre-registered information; detecting a weight of garbage dumped in through the dumping inlet; accumulating data of the detected weight into a memory for each of the users; and determining a schedule for inspection of the stirring mechanism and the deodorizing machine, and a schedule for regulation and replenishment of the bacteria, based on data of the garbage dumping date of at least one week inputted into the data entry unit and the accumulated data of the weight of at least one week, with reference to the maintenance program file.

In an eighth aspect of the present invention, a maintenance schedule creating method of a garbage disposal system comprising a stirring mechanism configured to stir dumped-in garbage together with bacteria and a deodorizing machine configured to deodorize odor from the garbage, the maintenance schedule creating method comprising: previously recording business type and scale of a user of the garbage disposal system into a user memory file; before dumping in garbage to be disposed of, inputting identification information of the user and a date on which the garbage is dumped in; opening a dumping inlet only in a case where the identification information coincides with pre-registered information; detecting, with a sensor, a weight of garbage dumped in through the dumping inlet; accumulating data of the detected weight into a memory for each of the users; and creating a schedule for inspection of the stirring mechanism and the deodorizing machine, and a schedule for regulation and replenishment of the bacteria, in a manner typified in accordance with the business type and scale of the user accumulated in the user memory file, based on the data of the garbage dumping date of at least one week and the accumulated data of the weight of at least one week.

In a ninth aspect of the present invention, a maintenance schedule creating method of a garbage disposal system comprising a stirring mechanism configured to stir dumped-in garbage together with bacteria and a deodorizing machine configured to deodorize odor from the garbage comprises: before dumping in garbage to be disposed of, inputting identification information of a user and a date on which the garbage is dumped in; opening a dumping inlet only in a case where the identification information coincides with pre-registered information; detecting, with a sensor, a weight of garbage dumped in through the dumping inlet and a load of the motor; accumulating data of the detected weight and load into a memory for each of the users, respectively; and determining a schedule for inspection of the stirring mechanism and the deodorizing machine, and a schedule for regulation and replenishment of the bacteria, based on the accumulated data of the weight and load of at least one week.

### Effects of the Invention

A garbage disposal system according to the present invention can create and execute a maintenance program based on disposal performance of at least one week, can accumulate and analyze data of the executed disposal to find a garbage disposal pattern that is more appropriate for actual conditions, can make the maintenance program more efficient, and can be maintained and inspected more precisely based on the program.

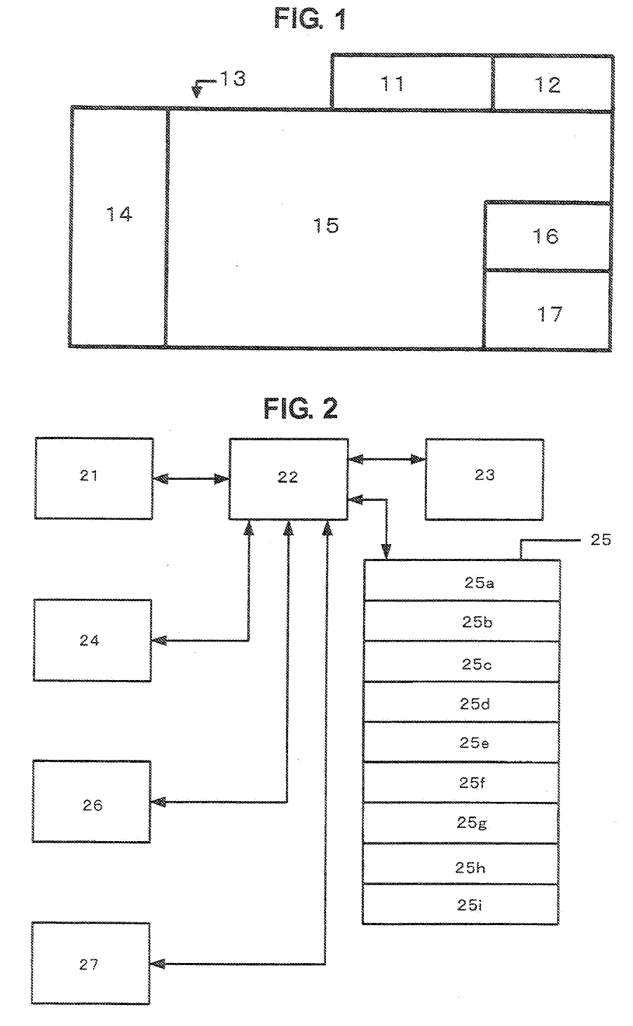

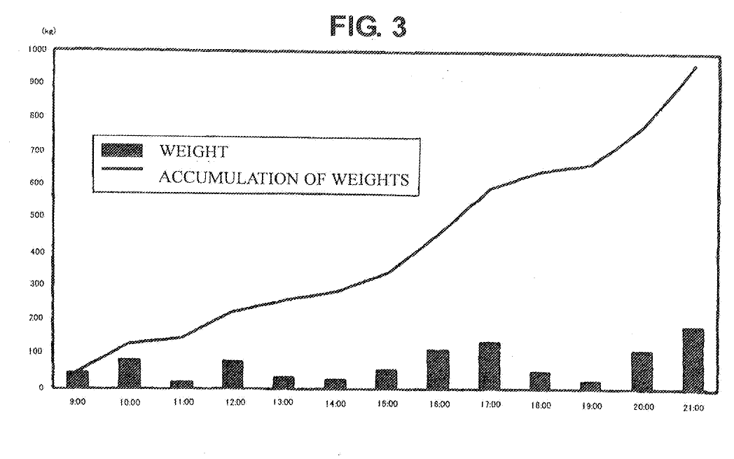

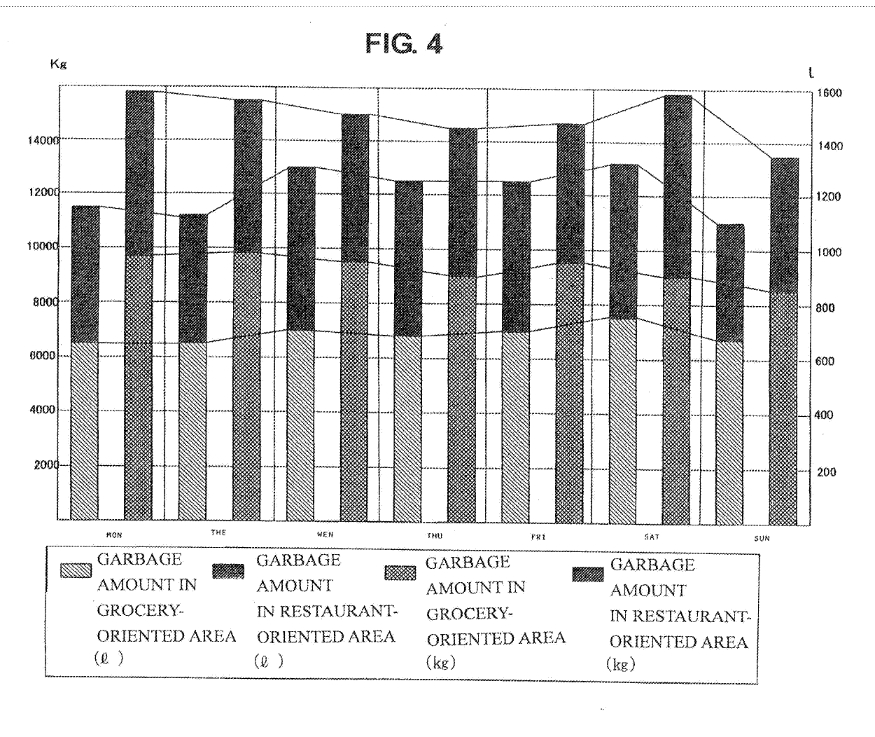

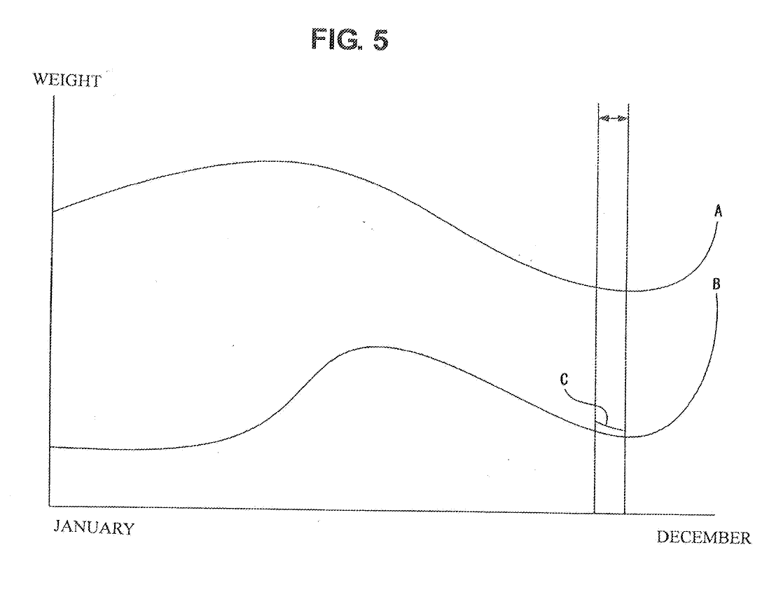

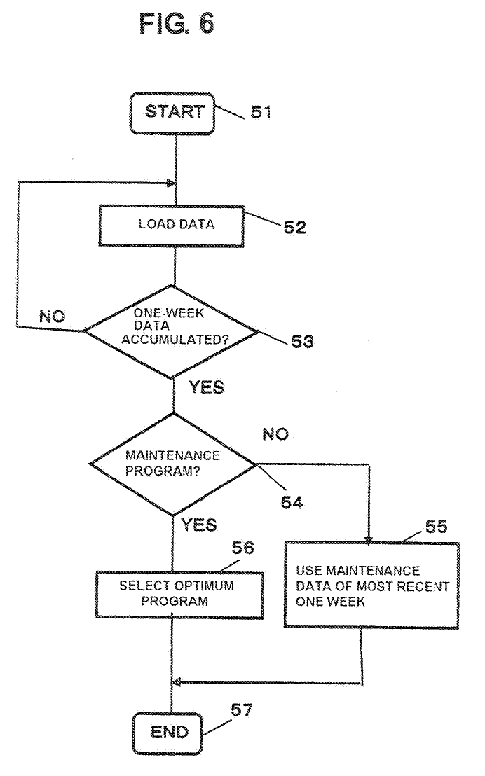

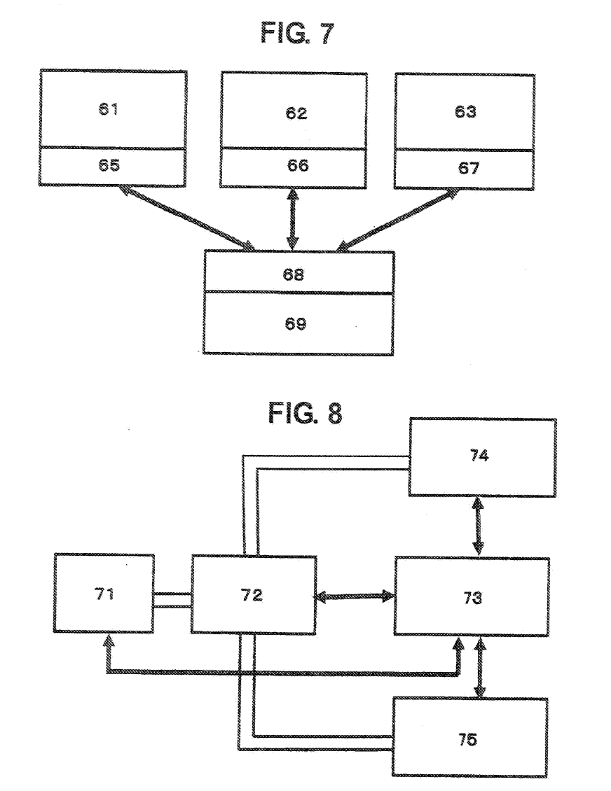

### BRIEF DESCRIPTION OF THE DRAWINGS

[FIG. 1] A block diagram of a garbage disposal system to which the present invention is applied

[FIG. 2] A block diagram of a controller in the garbage disposal system

[FIG. 3] A graph showing one example of a change with time in amount of dumped-in garbage within a day

[FIG. 4] A graph showing one example of a change depending on a day of the week in amount of dumped-in garbage within a week

[FIG. 5] A graph showing a type of a change pattern of the garbage amount

[FIG. 6] A flowchart of a garbage disposal system showing a first embodiment of the present invention

[FIG. 7] A block diagram of a garbage disposal system network showing a fourth embodiment of the present invention

[FIG. 8] A block diagram of a parallel garbage disposal system to which the present invention is applied

### EXPLANATION OF THE SYMBOLS

11 controller

12 dumping inlet

13 stirring tank

14 driving motor

15 stirring mechanism

16 deodorizing, machine

17 gas exhauster

21 data entry unit

22 processor

23 sensor unit

24 dumping inlet opening/closing unit

25 memory

26 display unit

27 printer

61 first garbage disposal system

62 second garbage disposal system

63 third garbage disposal system

65 modem

66 modem

67 modem

68 modem

69 integrated management system

71 crusher

72 switching valve

73 controller

74 first garbage disposal system

75 second garbage disposal system

### BEST EMBODIMENT TO IMPLEMENT THE INVENTION

An embodiment of the present invention will be described below referring to the drawings. FIG. 1 is a block diagram showing an embodiment of a garbage disposal system to which the present invention is applied. The garbage disposal system comprises a controller 11, a garbage dumping inlet 12, a stirring tank 13, a driving motor 14, a stirring mechanism 15, a deodorizing machine 16, and a gas exhauster 17.

In the garbage disposal system, garbage is dumped in through the garbage dumping inlet 12. The dumped-in garbage is conveyed to the stirring tank 13. The garbage is stirred in the stirring tank 13 by the stirring mechanism 15. Although not illustrated in the figure, the stirring mechanism 15 has a plurality of paddle-like arms arranged in the rotation axis direction, and at the tip of each of the arms, a spiral nail for mashing garbage is attached with a small gap from the inner wall of the stirring tank 13. Therefore, garbage is mashed efficiently when the stirring mechanism 15 is rotated by the driving motor 14. If it is preferred to crush before stir, a crusher may be installed.

The garbage mashed by the stirring mechanism 15 is stirred together with bacteria in the stirring tank 13, along with rotation of the stirring mechanism 15. The rotation of the stirring mechanism 15 is reversed, e.g., every 30 minutes so that the garbage is evenly disposed of, and the garbage is decomposed by the bacteria in 24 hours.

The bacteria may be bacteria in which, based on Bacillus bacteria living in the natural world like the soils, dried grasses and air, facultative anaerobic bacteria (amphoteric) and aerobic bacteria of cellulose decomposing bacteria, animal-fat decomposing bacteria, vegetable-fat decomposing bacteria, mineral-oil decomposing bacteria, protein decomposing bacteria, lignin decomposing bacteria (woody fungus) and the like are made to symbiotically live depending on disposals, or any known bacteria used for disposing of garbage. In the case of the former bacteria, garbage including wrapping materials such as plastic can be disposed of. Further, the combination and amount of the components of the bacteria are regulated depending on the proportion of food wastes and wrapping materials such as plastic. The bacteria with a porous ceramic bacterial bed are put in the stirring tank 13. The bacterial bed is approximately 15 mm in size, and is preferred to have not a granular shape but a shape with a sharp angle in order to promote finer crushing of garbage.

The garbage is decomposed by the decomposing action of the bacteria, and part thereof adheres to the internal wall of the stirring tank 13, but is scraped off by the spiral nail and collected as a residue to an exhaust part of the stirring tank 13, along with the rotation of the stirring mechanism 15.

Gas generated while the garbage is decomposed by the decomposing action of the bacteria is heated up to as high a temperature as 300° C., and is oxidized by a platinum catalyst with a honeycomb structure of the deodorizing machine 16. The deodorized gas is discharged in the air by the gas exhauster 17.

The residue can be utilized for a secondary processed product such as concrete. The volume reduction rate of the garbage reaches 97% to 98% and the garbage with the dumped-in volume of 100 littler is reduced to the residue of 2 litters to 3 litters. The volume reduction rate depends on the proportional ratio of food waste and plastic or the like wrapping the food waste.

FIG. 2 is a block diagram of the controller 11. The controller 11 comprises a data entry unit 21, a sensor unit 23, a dumping inlet opening/closing unit 24, a memory 25, a processor 22, a display unit 26, and a printer 27.

The controller 11 identifies a user, analyzes brought-in garbage, processes analysis data, and creates and executes a maintenance program. The processor 22 executes a program stored in the memory 25, creates various kinds of data, and controls the data entry unit 21, the sensor unit 23, the dumping inlet opening/closing unit 24, the memory 2, the display unit 26 and the printer 27, whereby the maintenance program is created and executed.

The data entry unit 21 reads identification information of the user inputted through the user's barcodes, credit cards, prepaid cards or entry keyboards. Since the users usually wear gloves to bring garbage into a garbage disposal system, it is favorable to previously stick a barcode onto a bag containing garbage so that the users can be identified. In a case where biometric authentication is employed, it is favorable to recognize a site other than a hand in consideration of wear of gloves.

The sensor unit 23 detects the weight of garbage, the temperature and humidity in the stirring tank 13, or the load of the driving motor 14. Furthermore, in order to obtain more accurate analysis data, it is desirable to install a sensor capable of detecting the volume and component of garbage, the pressure in the stirring tank 13, gas/gas component, or odor/odor component.

The dumping inlet opening/closing unit 24 controls the dumping inlet 12 to open and close. The dumping inlet opening/closing unit 24 opens the dumping inlet 12 only in a case where the identification information of the user read by the data entry unit 21 is the identification information of the registered user or is the identification information representing a maintenance inspector. Furthermore, the dumping inlet opening/closing unit 24 closes the dumping inlet 12 after a predetermined time elapses. Through the dumping inlet opening/closing, unit 24, the registered user dumps in garbage, and the maintenance inspector carries out maintenance.

The memory 25 stores a garbage data file 25a, a user data file 25b, a maintenance data file 25c, a garbage analysis program file 25d, a dumped-in garbage pattern file 25e, a maintenance program file 25f, a charge program file 25g, a related-information file 25h, and a control program file 25i.

The processor 22 executes control of the respective units 21, 23, 24, 25, 26 and 27 by executing a process in accordance with the control program file 25i. The display unit 26 and the printer 27 execute data display and output of data processing of the processor 22.

In the garbage data file 25a, garbage data outputted from the sensor 23 is accumulated. In the user data file 25b, data of names, scales, business types, registered dates and the like of the users are accumulated. In the maintenance data file 25c, information on what composition of bacteria has been replenished, which maintenance inspector has replenished bacteria, when and what composition of bacteria has been replenished, whether a bacterial bed has been replenished, and whether maintenance and regulation of mechanical parts have been carried out. In the related-information file 25h, reference data specific to an area, such as weather and population are previously accumulated. In the dumped-in garbage pattern file 25e, a change pattern analyzed for name, scale and business type of users, date and time, weather, or area is accumulated.

The garbage analysis program file 25d stores an analysis program. The processor 22 executes processing in accordance with the garbage analysis program file 25d, reads out and analyzes the garbage data file 25a and the user data file 25b or the related-information file 25h, makes a pattern from the accumulated changes according to the name, scale and business type of a garbage user, the date and time, the weather or the area, and outputs.

The maintenance program file 25f stores a maintenance program created in accordance with the dumped-in garbage pattern file 25e. In the maintenance program, the replenishment time, composition and amount of the bacteria, the replenishment time and amount of a bacterial bed, the setting of temperature and humidity inside the stirring tank 13, and the time of inspection such as maintenance and regulation of the components of the garbage disposal system such as the stirring mechanism 15 and the deodorizing machine 16 are described in accordance with the dumped-in garbage pattern file 25e.

The charge program file 25g stores a charge program. The processor 22 executes the charge program file 25g to charge the users mainly according to the weight of the dumped-in garbage.

The values of the usage fees are cumulated and accumulated in the user data file 25b for each of the users, whereby charging is performed. The values of fixed usage fees may be accumulated, but other than this, a time zone in which the amount of the dumped-in garbage is small may be analyzed by reading out the dumped garbage pattern file 25e and the values of the usage fees for the time zone may be accumulated. For example, it is possible to level the amount of dumped-in garbage over time zones by discounting the usage fee.

The identification information of the user read by the data entry unit 21 is collated with the user data file 25b of the memory 25 by the processor 22. When confirming that the user is a registered proper user through the collation, the processor 22 outputs a control signal for opening the dumping inlet 12 to the dumping inlet opening/closing unit 24.

When the dumping inlet 12 is opened and garbage is dumped in, the sensor unit 23 measures the weight and component of the dumped-in garbage. The sensor unit 23 outputs the obtained garbage data to the processor 22, and the processor 22 conducts a charging process for each of the users based on the garbage data in accordance with the charge program file 25g.

The accumulated values of the usage fees or the charged values are accumulated in the user data file 25b, and also displayed on a monitor by the display unit 26 and printed by the printer 27. Therefore, immediately after the garbage is dumped in, the user can check how much the garbage disposal cost is, from the monitor display data of the display unit 26 and an invoice issued from the printer 27.

FIG. 3 is one example of garbage data showing a change of garbage amount per hour in one day. FIG. 4 is one example of garbage data of garbage amount per day and per area for a week. Garbage amount changes dramatically depending on not only time of a day but also environmental conditions such as user's business type, scale, area, weather condition and season. Further, FIG. 5 is the dumped-in garbage pattern file 25e showing a type of a change pattern of garbage created based on accumulation of the garbage data shown in FIGS. 3 and 4, and shows the annual volume of dumped-in garbage per user. Reference symbol A indicates a change in garbage volume at western food restaurants, reference symbol B indicates a change in garbage volume at buckwheat noodle restaurants, and reference symbol C indicates a change in garbage volume for a week at a certain buckwheat noodle restaurant.

The inventor has found that it is possible to cyclically grasp a change for a day of the week in disposal amount and component of garbage brought in by a user in an area by at least one week unit and, since the amount of dumped-in garbage changes moderately depending on weather and season in the case of one week unit, it is possible to predict the amount of dumped-in garbage of the next one week based on garbage data of at least one week. Further, regarding types in which changes in garbage amount in one week are similar, the inventor has found that a correlation between a change pattern thereafter and a change in garbage amount thereafter. That is, the dumping process of garbage has a property of seven-state Markov process.

The processor 22 analyzes garbage data of at least one week and creates a schedule, i.e., a maintenance program of the next one week regarding time to inspect, time to taking out residues, time to replenish bacteria, composition of the bacteria, feeding amount of the bacteria, time to replenish bacterial bed and maintenance time of mechanism parts. For example, based on the dumped garbage pattern file 25e shown in FIG. 4, in the next one week, bacteria and bacterial bed are to be added on Wednesday and the temperature in the stirring tank is to be set on Monday.

For this, the garbage data from the sensor unit 23 for at least one week is accumulated in the memory 25, and a type with a similar change pattern is searched from the dumped garbage data file 25a and the dumped garbage pattern file 25e. Furthermore, from the searched-out change pattern type and the maintenance data file 25c, the maintenance program of the next week is created. As described above, in the maintenance data file 25c, the regular inspection, replacement and adjustment of mechanical parts, and the actual usage data of the combination and amount of bacteria are filed.

FIG. 6 is a flowchart showing an operation of creating a maintenance program by the garbage disposal system. The processor 22 of the garbage disposal system starts a process in accordance with the control program file 25i of the memory 25 in Step 51, and inputs garbage data from the sensor unit 23 into the garbage data file 25a of the memory 25 in Step 52.

Next, in Step 53, the processor checks whether the garbage data file 25a has accumulated the data of one week and, if not, continues inputting the garbage data. The processor 22 checks whether a maintenance program exists in the maintenance program file 25f in Step 54.

Then, in a case where a maintenance program does not exist in the maintenance program file 25f, in step 55, the processor uses the data already accumulated in the maintenance data file 25c to create a schedule regarding assignment of maintenance inspectors, replenishment of repair parts and bacteria, and preparation of bacterial bed, based on what day of the week, what time, what type of bacteria and bacterial bed need to be replenished and what type of mechanical part needs to be maintained and adjusted. The processor ends the process in Step 57.

On the other hand, in a case where a maintenance program exists in the maintenance program file 25f, in Step 56, the processor selects the most similar maintenance program in consideration of conditions such as business type scale of the user, disposal amount of garbage, timing, and weather conditions. The processor ends creation of the schedule in Step 57.

Based on the schedule created in this way, the garbage disposal state is checked, the residue is taken out, and replenishment of bacteria, replenishment of bacterial bed and maintenance of mechanical parts are conducted. An operator dedicated for handling the garbage disposal system performs the maintenance.

The garbage data daily accumulated and analyzed are used for maintenance in one week unit, and accumulated and analyzed together with environmental conditions such as user's business type, scale, area, weather conditions and seasons, thereby being classified into a dumped garbage pattern and stored in the dumped garbage pattern file 25e of the memory 25.

In a second embodiment of the present invention, if the dumped garbage pattern file 25e is ready in advance, the processor uses the garbage data of at least one week daily accumulated and analyzed, selects the most similar dumped garbage pattern from the dumped garbage pattern file 25e, and creates the maintenance program file 25f based on the pattern.

The processor 22 analyzes the garbage data of at least one week, searches a type with a similar change pattern from the dumped garbage pattern file 25e, and creates a schedule, i.e., a maintenance program of the next one week regarding time to inspect, time to take out residues, time to replenish bacteria, composition of the bacteria, feeding amount of the bacteria, time to replenish bacterial bed and maintenance time of mechanism parts.

Thus, it is possible to prepare long-term maintenance resources and short-term maintenance resources. In particular, in a case where the garbage disposal systems are installed in different places, it is possible to efficiently perform the assignment of maintenance inspectors, the replenishment of bacteria and bacterial bed, and the adjustment and maintenance of mechanical parts.

In a third embodiment of the present invention, the processor 22 analyzes data of at least one week accumulated in the garbage data file 25a based on the dumped garbage analysis program file 25d, estimates a pattern with respect to the user's business type and scale, and stores the pattern into the dumped garbage pattern file 25e as well as selects the most suitable maintenance program for the pattern from the maintenance program file 25f, thereby creating a maintenance schedule regarding the inspection, adjustment and replenishment of the stirring mechanism and bacteria depending on the dumped date/time and weight of garbage.

According to the third embodiment, even if the number of users of the garbage disposal system increases or decreases, it is possible to create the maintenance schedule by referring to the pattern based on the user's business type and scale stored in the dumped garbage pattern file 25e.

FIG. 7 is a network block diagram of a garbage disposal system showing a fourth embodiment of the present invention. In the network of this garbage disposal system, garbage disposal systems 61, 62 and 63 and an integrated management system 69 are provided with modems 65, 66, 67 and 78, respectively, for mutually transmitting/receiving data.

In this embodiment, the network is composed of three garbage disposal systems, but it is possible to connect more garbage disposal systems. Moreover, the garbage disposal systems 61, 62 and 63 may have different processing abilities.

The configurations of the garbage disposal systems 61, 62 and 63 are basically the same as the garbage disposal system shown in FIGS. 1 and 2, and each part will be described using the same reference symbols as in FIGS. 1 and 2 for convenience.

In the garbage disposal systems 61, 62 and 63, the same data processing is executed as in the above embodiment. Then, a maintenance schedule is created and maintenance is executed in each, and moreover, all data necessary for management is transmitted to the integrated management system 69 via the modems, and shared in the garbage disposal systems 61, 62 and 63.

The integrated management system 69 is composed of almost the same components as the controller of FIG. 2, and is provided with a processor 22, a memory 25, a display unit 26, and a printer 27.

The integrated management system 69 collects and accumulates data transmitted from the garbage disposal systems 61, 62 and 63, and creates the aforementioned dumped garbage pattern file 25e and maintenance program file 25f based on the garbage analysis program file 25d to transmit to the garbage disposal systems 61, 62 and 63. Consequently, each of the garbage disposal systems 61, 62 and 63 can search a maintenance program from a dumping pattern that is the most suitable therefor.

However, since a large amount of data needs to be accumulated for a long time period until the integrated management system 69 creates the dumped garbage pattern file 25e, each of the garbage disposal systems 61, 62 and 63 creates a maintenance schedule for the next one week based on performance data per at least one week until then. Further, in this case, for a long-term maintenance, common data is read out of the maintenance program file 25f and used.

The integrated management system 69 can transmit/receive data to/from the garbage disposal systems 61, 62 and 63 and manage when, what garbage disposal system executes maintenance to what extent, and what is required next.

In the above embodiment, data processing is executed mainly based on the weight of the dumped garbage. However, in the case of using data from the load sensor of the sensor unit 23 in addition, it is possible to create a maintenance program more efficiently and accurately because there is a correlation between the load sensor and the activity level of the bacteria in the stirring tank 13.

FIG. 8 is a block diagram of a parallel garbage disposal system to which the present invention is applied. The parallel garbage disposal system comprises a crusher 71, a switching valve 72, a controller 73, and first and second garbage disposal systems 74 and 75.

The controller 73 controls the crusher 71, the switching valve 72 and the first and second garbage disposal systems 74 and 75 of the parallel garbage disposal system. This controller has almost the same configuration as the controller of the above embodiment.

The crusher 71 is provided with a garbage dumping inlet 12, a dumping inlet opening/closing unit 24, a sensor unit 23 for measuring at least a weight, and a crushing mechanism. The weight of the dumped garbage is measured by the sensor unit 23, and the weight data of the garbage is outputted to the controller 73.

The controller 73 transmits/receives data to/from the first and second garbage systems 74 and 75, thereby being capable of grasping what weight of garbage is disposed of by the system when new garbage is dumped in.

Further, to the controller 73, load data of the driving motor 14 detected by the sensor unit 23 of each of the first and second garbage disposal systems 74 and 75 is outputted. Therefore, the processor 73 checks the disposing weights of the first and second garbage disposal systems 74 and 75, and operates the switching valve 72 so that the disposing weights become almost equal. If the disposing weights of the first and second garbage disposal systems 74 and 75 are almost equal, the switching valve 72 is controlled so that the loads become almost equal.

Thus, it is possible to increase the garbage disposing ability of the system by using a plurality of garbage disposal systems, and it is also possible to increase efficiency by controlling the disposing to be substantially equal.

In this system as well, it is possible to create a comprehensive maintenance schedule for the parallel garbage disposal system, based on the accumulated data for one week, as in the above embodiment.

As described above, the garbage disposal system according to the present invention has a significant advantage of being capable of creating and executing a maintenance program based on disposal performance of at least one week, accumulating and analyzing the process data thus executed, finding out a garbage disposal pattern as appropriate for the actual condition as possible, and making the maintenance program more efficient.

1.-21. (canceled)

22. A garbage disposal system configured to dispose of garbage by a decomposing action of bacteria, the garbage disposal system comprising:
a data entry unit to input identification information of a user and a date on which garbage is dumped in;
an opening/closing unit configured to open a dumping inlet only in a case where the identification information coincides with pre-registered information;
a sensor configured to detect a weight of garbage dumped in through the dumping inlet;
a stirring mechanism configured to stir the dumped-in garbage;
a deodorizing machine configured to deodorize odor from the garbage;
a memory to accumulate data of the weight detected by the sensor for each of the users recognized by the data entry unit; and
a processor configured to determine a schedule for inspection of the stirring mechanism and the deodorizing machine, and a schedule for regulation and replenishment of the bacteria, based on the data of the weight of at least one week accumulated in the memory.

23. The garbage disposal system according to claim 22, wherein:
the processor determines a schedule for inspection of the stirring mechanism and the deodorizing machine of next one week, and a schedule for regulation and replenishment of the bacteria of next one week.

24. The garbage disposal system according to claim 22, further comprising:
a garbage data file to accumulate data of the weight detected by the sensor for each of the users recognized by the data entry unit;
a maintenance program file configured to previously store a schedule for inspection of the stirring mechanism and the deodorizing machine, and a schedule for regulation and replenishment of the bacteria, in a manner separated into types in accordance with a garbage dumping date and weight; and
a maintenance processor configured to determine a schedule for next inspection of the stirring mechanism and the deodorizing machine, and a schedule for next regulation and replenishment of the bacteria, based on data of the garbage dumping date of at least one week inputted into the data entry unit and the weight of at least one week accumulated in the garbage data file, with reference to the maintenance program file.

25. The garbage disposal system according to claim 24, further comprising:
a user memory file to accumulate a business type and scale of the user; and
a processor configured to create a schedule for inspection of the stirring mechanism and the deodorizing machine, and a schedule for regulation and replenishment of the bacteria, in a manner separated into types in accordance with the business type and scale of the user accumulated in the user memory file, based on data of the garbage dumping date of at least one week inputted into the data entry unit and the weight of at least one week accumulated in the garbage data file.

26. The garbage disposal system according to claim 25 comprising:
a plurality of the garbage disposal machines, wherein:
in accordance with the business type and scale of the user accumulated in the user memory file or a group of the users, the processor analyzes the data of the weight accumulated in the garbage data file belonging to each of the garbage disposal machines, typifies the data of the garbage dumping date and weight, and creates the schedule for each of the types.

27. The garbage disposal system according to claim 22, wherein the stirring mechanism is provided with a motor,
the garbage disposal system further comprising:
a sensor configured to detect a weight of the garbage dumped in through the dumping inlet and a load of the motor;
a garbage data file to accumulate data of the weight and load detected by the sensor for each of the users recognized by the data entry unit, respectively; and
a processor configured to determine a schedule for inspection of the stirring mechanism and the deodorizing machine, and a schedule for regulation and replenishment of the bacteria, based on the data of the weight and load of at least one week accumulated in the garbage data file.

28. The garbage disposal system according to claim 27 further comprising:
a maintenance program file to store schedules for inspection of the stirring mechanism and the deodorizing machine and schedules for regulation and replenishment of the bacteria in a typified manner, wherein:
the processor determines a schedule for inspection of the stirring mechanism and the deodorizing machine and a schedule for regulation and replenishment of the bacteria, based on the data of the weight and load of at least one week accumulated in the garbage data file, from the types of the schedules for inspection of the stirring mechanism and the deodorizing machine and the schedules for regulation and replenishment of the bacteria stored in the maintenance program file.

29. The garbage disposal system according to claim 28 further comprising:
a maintenance program file configured to analyze the data of the weight and load accumulated in the garbage data file, and store a schedule for inspection of the stirring mechanism and the deodorizing machine and a schedule for regulation and replenishment of the bacteria in a typified manner; and
a user memory file to accumulate business type and scale of the user, wherein:
the processor selects a schedule in accordance with the data of the weight and load of at least one week accumulated in the garbage data file and the business type and scale of the user accumulated in the user memory file, from the types of the schedules for inspection of the stirring mechanism and the deodorizing machine and the schedules for regulation and replenishment of the bacteria stored in the maintenance program file.

30. A garbage disposal system comprising:
first and second garbage disposal systems, each including a stirring mechanism configured to stir garbage with a motor, a load sensor configured to detect a load of the motor, and a deodorizing machine configured to deodorize odor from garbage, and disposing of garbage by a decomposing action of bacteria;
a data entry unit to input identification information of a user and a date on which garbage is dumped in;
an opening/closing unit configured to open a dumping inlet only in a case where the identification information coincides with pre-registered information;
a weight sensor configured to detect a weight of garbage dumped in through the dumping inlet;
a crusher configured to crush the garbage dumped in through the dumping inlet;
a deodorizing machine configured to deodorize odor from the garbage;
a switching valve to convey the garbage crushed by the crusher to the first and second garbage disposal systems;
a garbage data file to accumulate data of the load detected by the load sensor and data of the weight detected by the weight sensor, respectively; and
a processor configured to control the switching valve based on the data of the load and weight accumulated in the garbage data file, substantially equalize the weights of the garbage disposed of by the first and second garbage disposal systems and the loads of the motors, and determine a schedule for inspection of the stirring mechanism and the deodorizing machine and a schedule for regulation and replenishment of the bacteria based on the data of the weight of at least one week accumulated in the garbage data file.

31. The garbage disposal system according to claim 22, wherein:
a display unit configured to display a result of execution of the processor is further provided.

32. The garbage disposal system according to claim 30, wherein:
a display unit configured to display a result of execution of the processor is further provided.

33. The garbage disposal system according to claim 22, wherein:
the data entry unit recognizes a garbage user from barcode data.

34. The garbage disposal system according to claim 30, wherein:
the data entry unit recognizes a garbage user from barcode data.

35. A maintenance schedule creating method of a garbage disposal system comprising a stirring mechanism configured to stir dumped-in, garbage together with bacteria and a deodorizing machine configured to deodorize odor from the garbage, the maintenance schedule creating method comprising:
before dumping in garbage to be disposed of, inputting identification information of a user and a date on which the garbage is dumped in;
opening a dumping inlet only in a case where the identification information coincides with pre-registered information;
detecting, with a sensor, a weight of garbage dumped in through the dumping inlet;
accumulating data of the detected weight into a memory for each of the users; and
determining a schedule for inspection of the stirring mechanism and the deodorizing machine, and a schedule for regulation and replenishment of the bacteria, based on the accumulated data of the weight of at least one week.

36. The maintenance schedule creating method according to claim 35, wherein:
a schedule for inspection of the stirring mechanism and the deodorizing machine of next one week and a schedule for regulation and replenishment of the bacteria of next one week are determined in the schedule-determining,

37. The maintenance schedule creating method according to claim 35, further comprising:
previously recording a schedule for inspection of the stirring mechanism and the deodorizing machine and a schedule for regulation and replenishment of the bacteria into a maintenance program, in a manner separated into types in accordance with a garbage dumping date and weight; and
determining a schedule for inspection of the stirring mechanism and the deodorizing machine, and a schedule for regulation and replenishment of the bacteria, based on data of the garbage dumping date of at least one week inputted into the data entry unit and the accumulated data of the weight of at least one week, with reference to the maintenance program file.

38. A maintenance schedule creating method of a garbage disposal system comprising a stirring mechanism configured to stir dumped-in garbage together with bacteria and a deodorizing machine configured to deodorize odor from the garbage, the maintenance schedule creating method comprising:
previously recording business type and scale of a user of the garbage disposal system into a user memory file;
before dumping in garbage to be disposed of, inputting identification information of the user and a date on which the garbage is dumped in;
opening a dumping inlet only in a ease where the identification information coincides with pre-registered information;
detecting, with a sensor, a weight of garbage dumped in through the dumping inlet;
accumulating data of the detected weight into a memory for each of the users; and
creating a schedule for inspection of the stirring mechanism and the deodorizing machine, and a schedule for regulation and replenishment of the bacteria, in a manner typified in accordance with business type and scale of the user accumulated in the user memory file, based on data of the garbage dumping date of at least one week and the accumulated data of the weight of at least one week.

39. The maintenance schedule creating method according to claim 38 relating to a plurality of the garbage disposal systems, wherein:
in the typifying, in accordance with the business type and scale of the user accumulated in the user memory file or a group of the users, the accumulated data of the weight belonging to each of the garbage disposal systems is analyzed, the data of the garbage dumping date and weight date arc typified, and a schedule is created for each of the types.

40. A maintenance schedule creating method of a garbage disposal system comprising a stirring mechanism configured to stir dumped-in garbage together with bacteria and a deodorizing machine configured to deodorize odor from the garbage, the maintenance schedule creating method comprising:
before dumping in garbage to be disposed of, inputting identification information of a user and a date on which the garbage is dumped in;
opening a dumping inlet only in a case where the identification information coincides with pre-registered information;
detecting, with a sensor, a weight of garbage dumped in through the dumping inlet and a load of the motor;
accumulating data of the detected weight and load into a memory for each of the users, respectively; and
determining a schedule for inspection of the stirring mechanism and the deodorizing machine, and a schedule for regulation and replenishment of the bacteria, based on the accumulated data of the weight and load of at least one week.

41. The maintenance schedule creating method according to claim 40 further comprising:
previously recording a schedule for inspection of the stirring mechanism and the deodorizing machine, and a schedule for regulation and replenishment of the bacteria, in a typified manner, wherein:
in the schedule-determining, a schedule for inspection of the stirring mechanism and the deodorizing machine and a schedule for the regulation and replenishment of the bacteria are determined based on the accumulated data of the weight and load of at least one week, from the types of the schedules for inspection of the stirring mechanism and the deodorizing machine and the schedules for regulation and replenishment of the bacteria previously recorded.

42. The maintenance schedule creating method of a garbage disposal system according to claim 40 further comprising:
previously recording business type and scale of the user, wherein:
in the schedule-determining, a schedule is selected in accordance with the data of the weight and load of at least one week accumulated in the garbage data file and the recorded business type and scale of the user, from the types of the schedule for inspection of the stirring mechanism and the deodorizing machine and the schedule for regulation and replenishment of the bacteria

43. The maintenance schedule creating method of a garbage disposal system according to claim 35 further comprising:
displaying an execution result in the schedule-determining on a display unit.

44. The maintenance schedule creating method of a garbage disposal system according to claim 38 further comprising:
displaying an execution result in the schedule-determining on a display unit.

45. The maintenance schedule creating method of a garbage disposal system according to claim 35, wherein:
in the inputting, a garbage user is recognized based on barcode data.

46. The maintenance schedule creating method of a garbage disposal system according to claim 38, wherein:
in the inputting, a garbage user is recognized based on barcode data.
